(19) United States
(12) Patent Application Publication
(10) Pub. No.: US 2021/0353751 A1
(43) Pub. Date: Nov. 18, 2021

(54) COMBINATION THERAPIES FOR TREATMENT OF INFLAMMATORY DISEASES

(71) Applicant: The Regents of The University of California, Oakland, CA (US)

(72) Inventors: Daniel Kaufman, Los Angeles, CA (US); Jide Tian, Los Angeles, CA (US)

(73) Assignee: The Regents of The University of California, Oakland, CA (US)

(21) Appl. No.: 17/286,030

(22) PCT Filed: Oct. 18, 2019
(86) PCT No.: PCT/US2019/056847

§ 371 Date: Apr. 16, 2021

Related U.S. Application Data
(60) Provisional application No. 62/747,370, filed on Oct. 18, 2018.
(60) Provisional application No. 62/747,398, filed on Oct. 18, 2018.

(51) Int. Cl. A61K 39/395; A61K 31/573; A61K 38/13; A61K 31/436; A61K 31/42; A61K 31/5377; A61K 31/407; A61K 31/519; A61K 31/5415; A61K 31/405; A61K 38/21; A61K 31/5513; A61K 31/515; A61K 31/145; A61K 31/197; A61K 31/351; A61K 31/401; A61K 31/05; A61K 31/437; A61P 3/10

(52) U.S. Cl. CPC A61K 39/3955; A61P 3/10; A61K 38/13; A61K 31/436; A61K 31/42; A61K 31/5377; A61K 31/407; A61K 31/519; A61K 31/5415; A61K 31/405; A61K 38/215; A61K 31/5513; A61K 31/515; A61K 31/145; A61K 31/197; A61K 31/351; A61K 31/401; A61K 31/05; A61K 31/437; A61K 31/573

(57) ABSTRACT

The present application relates to a combination therapy for treatment of inflammatory diseases comprising an immunomodulator compound and a GABA-receptor agonist in an amount effective to reduce inflammation and ameliorate disease. In certain embodiments, the present application relates to treatment of T1D by administering an immunomodulator compound and a GABA-receptor agonist in an amount effective to prevent, reduce, and/or treat hyperglycemia in the human or animal subject. In certain embodiments, the immunomodulator compound and GABA-receptor agonist are administered in an amount effective to control autoimmune responses and safely increase β-cell mass and function in the context of established β-cell autoimmunity.

### STATEMENT OF FEDERALLY-SPONSORED RESEARCH OR DEVELOPMENT

This invention was made with government support under Award AI119831, awarded by the National Institutes of Health. The government has certain rights in the invention.

### BACKGROUND OF THE INVENTION

Inflammation is an innate immune response that is part of an organism's natural defense against invading pathogens and trauma. During an inflammatory response, blood flow to the infected area increases, as does vascular permeability, thereby allowing numerous types of immune cells to enter the affected area. The immune cells that enter the area release a host of immunological compounds that further mediate the immune response.

While acute inflammation is a natural, and generally beneficial, immune response, there are also numerous diseases that are caused by or related to chronic or otherwise unchecked inflammatory reactions. These inflammatory diseases include, but are not limited to, Alzheimer's Disease, amyotrophic lateral sclerosis, asthma, atherosclerosis, cerebral abscess, cerebral ischaemia, Crohn's disease, encephalitis, hepatitis, inflammatory bowel disease, lupus, meningitis, migraines, multiple sclerosis, obesity, Parkinson's disease, periodontitis, rheumatoid arthritis, sarcoidosis, stroke, tuberculosis, ulcerative colitis, ulcers, and vasculitis, and type-1 diabetes. Inflammatory diseases can be painful and, in some cases, progressively debilitating, which can greatly affect one's quality of life and create both societal and economic burdens. Over 1 million people in the United States are living with multiple sclerosis (“MS”). Approximately 1.25 million people in the United States are living with type 1 diabetes (“T1D”). Over 1.3 million people in the United States have rheumatoid arthritis (“RA”). It has been reported that patients with chronic inflammatory conditions in the United States spend approximately $38,000 or more per year on additional expenditures. A study in 2010 found that annual costs for privately-insured and Medicare patients with rheumatoid arthritis were $306 million and $600 million, respectively. As many as 70,000 new cases of inflammatory bowel disease are diagnosed each year in the United States alone.

One inflammatory disease that is of great societal and economic importance is diabetes mellitus Type 1, which is also referred to as “Type 1 Diabetes” (“T1D”) and historically as “juvenile diabetes” and “insulin-dependent diabetes” (“IDDM”). T1D ultimately results from insufficient production of insulin by the pancreas. Though the exact cause of T1D remains unknown, the disease is known to have an inflammatory autoimmune component, during which the patient's immune system, which normally combats harmful bacteria and viruses, mistakenly destroys the insulin-producing cells in the pancreas, which are primarily the beta-cells (“β-cells”) in the islets of Langerhans (“islets”). Once a significant number of islet β-cells are destroyed, little or no insulin is produced. Insulin circulates and allows glucose, a sugar, to enter one's cells. Insufficient insulin production results in high blood sugar levels in the body. Symptoms of T1D include polyuria (increased urination), polydipsia (increased thirst), polyphagia (increased hunger), dry mouth, fatigue, weakness, irritability, blurred vision, and weight loss. Over time, T1D can negatively impact a number of major organs in the body, including the heart, blood vessels, nerves, eyes, and kidneys.

Islet transplantation offered hope as a curative measure for T1D, but more than 80% of transplanted islet cells die within one week after transplantation. Recently, the U.S. Food and Drug Administration approved the first “artificial pancreas” for patients with T1D. The device links a continuous glucose monitor to an insulin pump and automatically delivers the correct amount of insulin. While helpful, the artificial pancreas delivers only basal insulin, leaving bolus insulin following meals an issue, as well as concerns regarding the accuracy and reliability of the device.

There remains no cure for T1D. Controlling beta-cell (“β-cell”) autoimmunity and expanding β-cell mass are major goals for T1D therapy. Clinical trials have thus far not been able to prevent the eventual loss of β-cells and the corresponding loss of insulin production. Because T1D results from autoimmune-mediated destruction of insulin-producing β-cells, there remains a need to preserve both any residual β-cells, as well as newly-formed β-cells from the robust T-cell autoreactivity against β-cells.

Another inflammatory disease of great societal and economic importance is rheumatoid arthritis (“RA”). RA is a chronic autoimmune disorder that results in inflammation of the joints, producing swollen, painful and stiff joints. Like T1D, the exact cause is not fully eluciadated, but it is believed to result from a combination of genetic and environmental factors.heumatoid arthritis. There is currently no cure for RA. Treatment of RA primarily involves decreasing inflammation in the joints in order to reduce pain and swelling.

Multiple sclerosis (“MS”) is another inflammatory disease of great importance. MS is an autoimmune disease that involves demyelination of nerve cells. Again, the exact cause is not fully elucidated, but automimmunity and resulting inflammation ultimately lead to destruction of the myelin sheath, producing a host of central nervous system symptoms. There is currently no cure for MS.

During inflammatory responses, T cells infiltrate the area of inflammation. T cells express receptors for the nonprotein amino acid γ-aminobutyric acid (“GABA”). See J. Tian et al., GABA(A) receptors mediate inhibition of T cell responses, 96 J. NEUROIMMUNOL 21-28 (1999); J. Tian et al., Gamma-aminobutyric acid inhibits T cell autoimmunity and the development of inflammatory responses in a mouse type 1 diabetes model, 173 J. IMMUNOL 5298-5304 (2004); S. Alam et al., Human peripheral blood mononuclear cells express GABAA receptor subunits, 43 MOL. IMMUNOL 1432-1442 (2006); S. K. Mendu et al., Different subtypes of GABA-A receptors are expressed in human, mouse and rat T lymphocytes, 7 PLoS ONE e42959 (2012); G. J. Prud'homme et al., GABA Protects Human Islet Cells Against the Deleterious Effects of Immunosuppressive Drugs and Exerts Immunoinhibitory Effects Alone, 96 TRANSPLANTATION 616-623 (2013). There are two types of GABA-receptors (“GABA-Rs”) that are encoded by distinct gene families and their activation induces different pathways—GABA_{A}-Rs are fast-acting chloride channels and GABA_{B}-Rs are slow-acting G-protein coupled receptors. See R. W. Olsen et al., Molecular biology of GABAA receptors, 4 FASEB J 1469-1480 (1990); B. Bettler et al., Molecular structure and physiological functions of GABA(B) receptors, 84 PHYSIOL REV 835-867 (2004). Rodent and human T cells expresses functional GABA_{A}-Rs but appear unresponsive to GABA_{B}-R-specific agonists. See J. Tian et al., GABA(A) receptors mediate inhibition of T cell responses, 96 J. NEUROIMMUNOL 21-28 (1999); S. Alam et al., Human peripheral blood mononuclear cells express GABAA receptor subunits, 43 MOL IMMUNOL 1432-1442 (2006); S. K. Mendu et al., Different subtypes of GABA-A receptors are expressed in human, mouse and rat T lymphocytes, 7 PLoS ONE e42959 (2012); G. J. Prud′homme et al., GABA protects human islet cells against the deleterious effects of immunosuppressive drugs and exerts immunoinhibitory effects alone, 96 TRANSPLANTATION 616-623 (2013). In mice, GABA administration limited delayed-type hypersensitivity (“DTH”) responses and inhibited or reversed disease in models of T1D (see Tian et al., GABA(A) receptors mediate inhibition of T cell responses, 96 J. NEUROIMMUNOL 21-28 (1999); J. Tian et al., Gamma-aminobutyric acid inhibits T cell autoimmunity and the development of inflammatory responses in a mouse type I diabetes model, 173 J. IMMUNOL 5298-5304 (2004); G. J. Prud′homme et al., GABA protects human islet cells against the deleterious effects of immunosuppressive drugs and exerts immunoinhibitory effects alone, 96 TRANSPLANTATION 616-623 (2013); N. Soltani et al., GABA exerts protective and regenerative effects on islet beta cells and reverses diabetes, 108 PROC. NATL. ACAD. SCI. 11692-11697 (2011); S. K. Mendu et al., Increased GABA(A) channel subunits expression in CD8(+) but not in CD4(+) T cells in BB rats developing diabetes compared to their congenic littermates, 48 MOL. IMMUNOL 399-407 (2011); J. Tian et al., Combined therapy with GABA and proinsulin/alum acts synergistically to restore long-term normoglycemia by modulating T-cell autoimmunity and promoting beta-cell replication in newly diabetic NOD mice, 63 DIABETES 3128-3134 (2014)), rheumatoid arthritis (see J. Tian et al., Oral GABA treatment downregulates inflammatory responses in a mouse model of rheumatoid arthritis, 44 AUTOIMMUNITY 465-470 (2011)), and limited inflammation and disease in type 2 diabetes models. See J. Tian et al., Oral treatment with gamma-aminobutyric acid improves glucose tolerance and insulin sensitivity by inhibiting inflammation in high fat diet-fed mice, 6 PLoS ONE e25338 (2011); S. Sohrabipour et al., GABA dramatically improves glucose tolerance in streptozotocin-induced diabetic rats fed with high-fat diet, EUR J PHARMACOL 2018.01.047 (2018). Studies of the mechanisms underlying those observations revealed that GABA treatment inhibited the development of autoreactive Th1 responses while also promoting CD4⁺ regulatory T cells (“Tregs”). These preclinical studies provided the basis for an ongoing clinical trial in which GABA is being given to individuals newly diagnosed with T1D (NCT02002130).

There remains a need for improved treatments for inflammatory diseases, including T1D, RA, and MS.

### SUMMARY OF THE INVENTION

Provided are new combination therapies to reduce and/or inhibit inflammation and thereby ameliorate inflammatory disease. The inventors have demonstrated that a combination of a GABA-receptor agonist and an immunomodulator, such as an immunosuppressant, can treat inflammatory disease, including at low dosages of the immunomodulator, such as by effectively reducing hyperglycemia in newly-diabetic animals and thereby ameliorating T1D.

In one embodiment, the present application relates to methods for treating inflammatory disease in a human or animal subject in need thereof, the method comprising administering to the human or animal subject one or more immunomodulators and one or more GABA-receptor agonists in an amount effective to ameliorate said inflammatory disease. In other embodiments, the one or more immunomodulator compounds and one or more GABA-receptor agonists are administered in an amount effective to reduce inflammation.

In certain embodiments, the inflammatory disease is selected from the group consisting of type-1 diabetes, rheumatoid arthritis, Alzheimer's Disease, amyotrophic lateral sclerosis, asthma, atherosclerosis, cerebral abscess, cerebral ischaemia, Crohn's disease, encephalitis, hepatitis, inflammatory bowel disease, lupus, meningitis, migraines, multiple sclerosis, obesity, Parkinson's disease, periodontitis, rheumatoid arthritis, sarcoidosis, stroke, tuberculosis, ulcerative colitis, ulcers, and vasculitis, and type-1 diabetes. In specific examples, the inflammatory disease is type-1 diabetes. In other examples, the one or more immunomodulator compounds and one or more GABA-receptor agonists are administered in an amount effective to prevent, reduce, and/or treat hyperglycemia and/or improve blood glucose levels in a human or animal subject suffering from T1D.

In certain embodiments that subject is an adult or juvenile human. In other embodiments, the subject is a companion animal, such as a dog, cat, rabbit, or horse.

In further embodiments, the one or more immunomodulator compounds comprise one or more immunoregulators, immunostimulants, or immunosuppressants. In particular examples, the one or more immunomodulator compounds comprise one or more immunosuppressants. In certain examples, the one or more immunomodulator compounds are selected from the group consisting of an anti-CD3 immunotherapy compound, corticosteroids, prednisone, budesonide, prednisolone, methylprednisolone, calcineurin inhibitors, cyclosporine, tacrolimus, mTOR inhibitors, sirolimus, everolimus, IMDH inhibitors, azathioprine, leflunomide, mycophenolate, biologics, abatacept, adalimumab, anakinra, certolizumab, etanercept, golimumab, infliximab, ixekizumab, natalizumab, rituximab, secukinumab, tocilizumab, ustekinumab, vedolizumab, monoclonal antibodies, basiliximab, daclizumab, muromonab, anti-lymphocyte globin, anti-thymocyte globin, lymphocyte immune globulin, thymoglobulin, mycophenolate mofetil, mycophenolate sodium, glucocorticoids, NSAIDs/COX inhibitors (e.g., aspirin, celecoxib, diclofenac, diflunisal, etodolac, ibuprofen, indomethacin, ketoprofen, ketorolac, nabumetone, naproxen, oxaprozin, piroxicam, salsalate, sulindac, and tolmetin), methotrexate, hydroxychloroquine, sulfasalazine, copaxone, and interferon β. In particular examples, the one or more immunomodulator compounds comprise an anti-CD3 immunotherapy compound. In other examples, the anti-CD3 immunotherapy compound comprises non-Fc binding anti-CD3ε Fab.

In other embodiments, the GABA-receptor agonist is selected from the group consisting of thiopental, thiamylal, pentobarbital, secobarbital, hexobarbital, butobarbital, amobarbital, barbital, mephobarbital, phenobarbital, primidone, midazolam, triazolam, lometazepam, flutazolam, nitrazepam, fluritrazepam, nimetazepam, diazepam, medazepam, oxazolam, prazeam, tofisopam, rilmazafonoe, lorazepam, temazepam, oxazepam, fluidazepam, chlordizaepoxide, cloxazolam, flutoprazepam, alprazolam, estazolam, bromazepam, flurazepam, clorazepate potassium, haloxazolam, ethyl loflazepate, qazepam, clonazepam, mexazolam, etizolam, brotizolam, clotizaepam, propofol, fospropofol, zolpidem, zopiclone, exzopiclone, muscimol, THIP/gaboxadol, Isoguvacine, Kojic amine, GABA, Homotaurine, Homohypotaurine, Trans-aminocyclopentane-3-carboxylic acid, Trans-amino-4-crotonic acid, β-guanidinopropionic acid, homo-β-proline, Isonipecotic acid, 3-((aminoiminomethyl)thio)-2-propenoic acid (ZAPA), Imidazoleacetic acid, and piperidine-4-sulfonic acid (P4S). In certain examples, the GABA-receptor agonist is GABA.

In further embodiments, one or both of the immunomodulator compounds and GABA-receptor agonists are administered at a subclinical and/or suboptimal dose of the compound when administered as a monotherapy.

In particular embodiments, the immunomodulator is anti-CD3 administered intravenously in an amount of 0.2-20 mg/kg/day. In particular examples, the anti-CD3 is administered in amount of 0.4-15 mg/kg/day, 0.6-10 mg/kg/day, 1-5 mg/kg/day, or 1-4 mg/kg/day.

In other embodiments, the GABA-receptor agonist is GABA administered intraperitoneally in an amount of 1 ng/kg/day to 1 g/kg/day. In particular examples, the GABA is administered intraperitoneally in amount of 1 ng/kg/day-500 mg/kg/day, 10 ng/kg/day-500 mg/kg/day, 50 ng/kg/day-500 mg/kg/day, 100 ng/kg/day-500 mg/kg/day, 200 ng/kg/day-500 mg/kg/day, 400 ng/kg/day-250 mg/kg/day, 750 ng/kg/day-100 mg/kg/day, 1-1000 μg/kg/day 50-1500 μg/kg/day, 100-1000 μg/kg/day, 150-500 μg/kg/day, or 200-400 μg/kg/day. In other embodiments, the GABA-receptor agonist is GABA administered orally in an amount of 100-10,000 mg/kg/day. In particular examples, the GABA is administered orally in amount of 500-5000 mg/kg/day, 1000-4000 mg/kg/day, or 2000-3000 mg/kg/day. In still further embodiments, the GABA-receptor agonist is homotaurine administered orally in an amount of 10-500 mg/kg/day. In particular examples, the homotaurine is administered orally in amount of 12-300 mg/kg/day, 14-200 mg/kg/day, 16-150 mg/kg/day, or 25-100 mg/kg/day.

### BRIEF DESCRIPTION OF THE DRAWINGS

FIG. 1 depicts representative islets containing bi-hormonal (glucagon⁺ insulin⁺) islet-cells in control non-obese diabetic (“NOD”) mice who received anti-CD3 and either plain water or phosphate-buffered saline (“PBS”) intraperitoneally (“IP”). White arrows, magnification×400, white scale bar is 25 μm.

FIG. 2 depicts representative islets with many β-cells surrounded by benign insulitis in NOD mice who received anti-CD3 and GABA. White arrows, magnification×200, white scale bar is 25 μm.

FIG. 3 depicts a quantitative analysis of bihormonal glucagon⁺ insulin⁺ cells.

FIG. 4 depicts a quantitative analysis of the percentages of β-cells in all islets.

FIG. 5 provides an analysis of the proportion of newly-diabetic NOD mice that remained normoglycemic across a 25 week study in which the mic were administered anti-CD3 (circle), anti-CD3+homotaurine (triangle), or anti-CD3+GABA (square).

FIG. 6 depicts a homotaurine dose-finding study and a combined homotaurine treatment with proinsulin/alum in newly diabetic NOD mice. In pilot studies, newly diabetic NOD mice were untreated (FIG. 6A), or continually given homotaurine at 0.08 mg/ml (n=6) (FIG. 6B), 0.25 mg/ml (n=9) (FIG. 6C), or 0.75 mgs/ml (n=12) (FIG. 6D) through their drinking water. Subsequently, another group of newly diabetic NOD mice received both homotaurine (0.25 mg/ml) and proinsulin/alum immunization (n=9) (FIG. 6E). Data shown are longitudinal blood glucose levels for individual mice. Dashed line indicates blood glucose of 250 mg/dL. The percentage of mice in each treatment group that remained relapse free over a 45 week period are shown in FIG. 6F.

FIG. 7 depicts the results of combined homotaurine and low-dose anti-CD3 on hyperglycemia in severely diabetic NOD mice. Newly diabetic NOD mice with severe hyperglycemia were given homotaurine (0.25 mg/ml, n=7; FIG. 7A), low-dose anti-CD3 (n=13; FIG. 7B), or combined low dose anti-CD3+homotaurine (n=25; FIG. 7C). FIG. 7D depicts the percentage of relapse-free mice treated with homotaurine (triangle symbol), low-dose anti-CD3 (circle symbol), or combined low dose anti-CD3+homoaurine (square symbol) over the 25 week period. Statistical analysis indicates: Homotaurine vs. anti-CD3+homotaurine (p=0.002) and anti-CD3 vs. anti-CD3+homotaurine (p=0.05) by the log-rank test.

### DETAILED DESCRIPTION OF THE INVENTION

The inventors have demonstrated that a combination of a GABA-receptor agonist and an immunomodulator, such as an immunosuppressant, can treat inflammatory disease, including at low dosages of the immunomodulator. In certain embodiments, the inflammatory disease is T1D and the one or more immunomodulator compounds and one or more GABA-receptor agonists are administered in an amount effective to prevent, reduce, and/or treat hyperglycemia and/or improve blood glucose levels in a human or animal subject suffering from T1D.

Murine and human insulin-producing β-cells and glucagon-expressing α-cells express gamma-aminobutyric acid (“γ-aminobutyric acid” or “GABA”) receptors (“GABA-Rs”). It has been shown that the activation of the β-cell receptors can promote β-cell survival and replication. Past studies showed that orally delivered GABA monotherapy in newly-diabetic NOD mice briefly delayed progression from moderate to severe hyperglycemia.¹ Treatment with oral GABA at 20 mg/ml restored normoglycemia in all mice, but all mice became hyperglycemic again, generally within five weeks. Following treatment with a combined proinsulin/alum immunization and GABA at 20 mg/ml, all mice became normoglycemic for at least thirteen weeks. However, most mice reverted to hyperglycemia 13-24 weeks after initiation of treatment, with 22% of the mice remaining normoglycemic for the duration of the study (45 weeks post initiation of treatment).¹ Thus, while the combination of antigen-specific immunotherapy and GABA was able to restore normoglycemia, the remission was not long-term in most animals.

Within the nervous system, GABA can promote neurogenesis and neuronal proliferation and is a neuronal survival factor. GABA has previously been analyzed for its ability to reduce seizures in hundreds of epilepsy patients participating in long-term clinical trials. Unfortunately, this therapy showed no clinical benefit. The lack of clinical benefit may have been due to the inability of GABA to cross the blood-brain barrier. However, the inability of GABA to transverse the blood-brain barrier makes it an ideal candidate to modulate peripheral GABA-receptors with minimal to no CNS side effects. Using GABA to limit autoimmune responses as well as promote B-cell survival and replication in the periphery is therefore a safe and highly attractive proposition.

GABA receptor activation inhibits pathogenic T-cell responses, induces regulatory T-cells, and can prevent T1D, experimental autoimmune encephalomyelitis (“EAE”), and rheumatoid arthritis in mouse models. GABA inhibits antigen-induced human T-cell proliferation in vitro, indicating that GABA receptors are expressed by human immunocompetent cells. Moreover, GABA treatment also promotes mouse and human B-cell survival and replication, and can increase human B-cell mass in human islets implanted into immune-deficient mice.

There is speculation that GABA can convert α-cells into β-like cells.² GABA may also start neogenesis of new islet cells and new α-cells may then be converted into β-like cells. When β-cells self-replicate, they are true “β-cells.” When α-cells convert into β-cells, they may still retain some α-cell features, and are thus collectively referred to as “β-like cells.” Those studies implied that long-term GABA treatment may lead to complete replenishing of insulin-producing cells in non-autoimmune mice in which no immunosuppressants are needed to protect residual β-cells, β-cells that arise subsequently through self-replication, or β-like cells that arise subsequently due to GABA treatment.² Recent studies, however, indicate that those findings were not reproducible. Moreover, those studies were conducted in non-autoimmune mice, so nothing was required to protect new β-cells or new β-like cells from autoreactivity. Contrarily, under autoimmune conditions, the autoimmune response will quickly destroy new β-cells or new β-like cells.

In an effort to reduce inflammation and restore normoglycemia, the present inventors administered a combination of an immunomodulator compound and a GABA-receptor agonist to newly-diabetic NOD mice. This combination effectively reversed hyperglycemia and increased β-cells in newly-diabetic NOD mice, which have robust autoimmunity to β-cells. Thus, the present inventors surprisingly demonstrated that the combination of a treatment that controls autoreactivity (an immunomodulator) with a treatment that can promote β-cell mass (a GABA-receptor agonist) can enable sufficient replenishment of β-cells and/or β-like cells in those developing T1D, or those with T1D. This approach, which successfully controls pathogenic T-cell autoimmunity and also promotes β-cell replenishment, is a major advance towards preventing and reducing the complications associated with T1D.

In one embodiment, the present application relates to methods for treating inflammatory disease in a human or animal subject in need thereof, the method comprising administering to the human or animal subject one or more immunomodulators and one or more GABA-receptor agonists in an amount effective to ameliorate said inflammatory disease. In other embodiments, the one or more immunomodulator compounds and one or more GABA-receptor agonists are administered in an amount effective to reduce inflammation.

In certain embodiments, the inflammatory disease is selected from the group consisting of type-1 diabetes, rheumatoid arthritis, Alzheimer's Disease, amyotrophic lateral sclerosis, asthma, atherosclerosis, cerebral abscess, cerebral ischaemia, Crohn's disease, encephalitis, hepatitis, inflammatory bowel disease, lupus, meningitis, migraines, multiple sclerosis, obesity, Parkinson's disease, periodontitis, rheumatoid arthritis, sarcoidosis, stroke, tuberculosis, ulcerative colitis, ulcers, and vasculitis, and type-1 diabetes. In specific examples, the inflammatory disease is type-1 diabetes.

In certain examples, the one or more immunomodulator compounds and one or more GABA-receptor agonists are administered in an amount effective to prevent, reduce, and/or treat hyperglycemia in the human or animal subject suffereing from T1D. In certain embodiments, a prevention, reduction, and/or treatment of hyperglycemia can be determined by reduced or improved blood glucose level. Blood glucose level can be determined by using a conventional blood glucose test, which would be well-known to one of ordinary skill in the art. In other embodiments, a prevention, reduction, and/or treatment of hyperglycemia can be identified as a decreased insulin requirement, increased HbA1c level, and/or increased C-peptide measurement in the subject being treated.

In certain embodiments, the one or more immunomodulators and one or more GABA-receptor agonists are administered in an amount effective to positively impact the number or percentage of insulin-producing β-cells in the islets of the pancreas. In particular embodiments, the immunomodulator compounds are administered in an amount effective to reduce or delay the elimination of insulin-producing β-cells from the islets of the pancreas. In other embodiments, the one or more GABA-receptor agonists are administered in an amount effective to increase the number and/or percentage of insulin-producing cells in the islets of the pancreas.

In certain embodiments the subject is an adult or juvenile human. In other embodiments, the subject is a companion animal, such as a dog, cat, rabbit, or horse.

As used herein, an “immunomodulator” is a compound that mediates (e.g., induces, enhances, or suppresses) an immune response, interferes with immune cell activation, or induces immune cell anergy or deletion. Immunomodulators that elicit or amplify immune responses are referred to as activation immunomodulators or immunostimulants, whereas immunomodulators that reduce or suppress immune responses are referred to as suppression immunomodulators or immunosuppressants.

In certain embodiments, the one or more immunomodulator compounds comprise one or more immunostimulants or immunosuppressants. In particular examples, the one or more immunomodulator compounds comprise one or more immunosuppressants. In other examples, the one or more immunomodulator compounds comprise one or more immunostimulants.

Immunomodulators that may be employed include: anti-CD3; corticosteroids, such as prednisone, budesonide, prednisolone, and methylprednisolone; calcineurin inhibitors, such as cyclosporine and tacrolimus; mTOR inhibitors, such as sirolimus and everolimus; IMDH inhibitors, such as azathioprine, leflunomide, and mycophenolate; biologics, such as abatacept, adalimumab, anakinra, certolizumab, etanercept, golimumab, infliximab, ixekizumab, natalizumab, rituximab, secukinumab, tocilizumab, ustekinumab, and vedolizumab; monoclonal antibodies, such as basiliximab, daclizumab, and muromonab; anti-lymphocyte globin; anti-thymocyte globin; lymphocyte immune globulin; anti-thymoglobulin; mycophenolate mofetil; mycophenolate sodium; glucocorticoids; NSAIDs/COX inhibitors, such as aspirin, celecoxib, diclofenac, diflunisal, etodolac, ibuprofen, indomethacin, ketoprofen, ketorolac, nabumetone, naproxen, oxaprozin, piroxicam, salsalate, sulindac, and tolmetin; methotrexate, hydroxychloroquine; sulfasalazine, copaxone, and interferon (3.

In particular examples, the one or more immunomodulator compounds comprise an anti-CD3 immunotherapy compound. In other examples, the anti-CD3 immunotherapy compound comprises non-Fc binding anti-CD3ε Fab. Anti-CD3 has been shown to slow the loss of c-peptide level in T1D clinical trials.^{3, 4, 5, 6} Anti-CD3 fails to maintain normoglycemia in newly-diabetic individuals, perhaps due to chronic exhaustion of the remaining β-cells, the lack of sufficient β-cell regeneration, and/or insufficient suppression of autoimmunity.

In certain embodiments, the immunomodulator is administered intravenously, subcutaneously, intraperitoneally, intramuscularly, or orally.

In certain examples, the immunomodulator compound is administered intravenously, subcutaneously, intraperitoneally, or intramuscularly in an amount of 0.01-500 mg/kg/day. In other examples, the immunomodulator compound is administered intravenously, subcutaneously, intraperitoneally, or intramuscularly in an amount of at least 0.01, 0.02, 0.05, 0.1, 0.25, 0.5, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 120, 140, 160, 180, 200, 225, 250, 275, 300, 350, 400, 450, or 500 mg/kg/day. In other examples, the immunomodulator is administered intravenously, subcutaneously, intraperitoneally, or intramuscularly in an amount of not more than 0.01, 0.02, 0.05, 0.1, 0.25, 0.5, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 120, 140, 160, 180, 200, 225, 250, 275, 300, 350, 400, 450, or 500 mg/kg/day. In further examples, the immunomodulator is administered in a dosage range comprising any of the upper and lower limits described herein. In still further examples, the immunomodulator is administered orally in an amount that is 1-1000 times that of the dosages listed above for intravenous, subcutaneous, intraperitoneal, or intramuscular administration.

In particular embodiments, the immunomodulator is anti-CD3 administered intravenously at a dose of 0.2-20 mg/kg/day. In particular examples, the anti-CD3 is administered at a dose of 0.4-15 mg/kg/day, 0.6-10 mg/kg/day, 1-5 mg/kg/day, or 1-4 mg/kg/day.

In other embodiments, the immunomodulator is antithymocyte globulin (ATG) administered intravenously at a dose of 1-10 mg/kg. In particular examples, ATG is administered at a dose of 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, or 10 mg/kg.

In further embodiments, the immunomodulator is Alprazolam administered intraperitoneally at a dose of 0.05-10 mg/kg/day. In particular examples, Alprazolam is administered at a dose of 0.05 mg/kg/day, 0.1 mg/kg/day, 0.15 mg/kg/day 0.2 mg/kg/day, 0.25 mg/kg/day, 0.3 mg/kg/day, 0.35 mg/kg/day, 0.4 mg/kg/day, 0.45 mg/kg/day, 0.5 mg/kg/day, 0.55 mg/kg/day, 0.6 mg/kg/day, 0.65 mg/kg/day, 0.7 mg/kg/day, 0.75 mg/kg/day 0.8 mg/kg/day, 0.85 mg/kg/day, 0.9 mg/kg/day, 0.95 mg/kg/day, 1 mg/kg/day, 1.1 mg/kg/day, 1.25 mg/kg/day, 1.5 mg/kg/day 1.75 mg/kg/day, 2 mg/kg/day, 2.5 mg/kg/day, 3 mg/kg/day, 3.5 mg/kg/day, 4 mg/kg/day, 4.5 mg/kg/day, 5 mg/kg/day, 6 mg/kg/day, 7 mg/kg/day, 8 mg/kg/day, 9 mg/kg/day, or 10 mg/kg/day.

In certain embodiments, the immunomodulator is administered 1, 2, 3, 4, 5, or 6 times per day. In other embodiments, the immunomodulator is administered every day, every other day, every 3 days, every 4, days, every 5 days, every 6 days, every 7 days, every 8 days, every 9 days, every 10 days, every 11 days, every 12 days, every 13 days, every 14 days, once per week, twice per week, 3 times per week, monthly, twice per month, 3 times per month, or 4 times per month. In further embodiments, the immunomodulator is administered for 1, 2, 3, 4, 5, 6, 7, 8, 9, 10, 11, 12, 13, or 14 days or for 1, 2, 3, 4, 5, 6, 7, 8, 9, 10, 11, or 12 months or more.

GABA receptor agonists that may be employed include: thiopental, thiamylal, pentobarbital, secobarbital, hexobarbital, butobarbital, amobarbital, barbital, mephobarbital, phenobarbital, primidone, midazolam, triazolam, lometazepam, flutazolam, nitrazepam, fluritrazepam, nimetazepam, diazepam, medazepam, oxazolam, prazeam, tofisopam, rilmazafonoe, lorazepam, temazepam, oxazepam, fluidazepam, chlordizaepoxide, cloxazolam, flutoprazepam, alprazolam, estazolam, bromazepam, flurazepam, clorazepate potassium, haloxazolam, ethyl loflazepate, qazepam, clonazepam, mexazolam, etizolam, brotizolam, clotizaepam, propofol, fospropofol, zolpidem, zopiclone, exzopiclone, muscimol, THIP/gaboxadol, Isoguvacine, Kojic amine, GABA, Homotaurine, Homohypotaurine, Trans-aminocyclopentane-3-carboxylic acid, Trans-amino-4-crotonic acid, β-guanidinopropionic acid, homo-β-proline, Isonipecotic acid, 3-((aminoiminomethyl)thio)-2-propenoic acid (ZAPA), Imidazoleacetic acid, and piperidine-4-sulfonic acid (P4S). In certain examples hybrid polypeptides of two GABA-receptor agonists can be used. In certain embodiments the immunomodulator compound and/or the GABA-receptor agonist may be administered in adjuvants, such as alum, to help induce regulatory responses to the antigen.

In certain embodiments, the GABA-receptor agonist is administered intravenously, subcutaneously, intraperitoneally, intramuscularly, or orally.

In certain examples, the GABA-receptor agonist compound is administered intravenously, subcutaneously, intraperitoneally, or intramuscularly in an amount of 1 ng/kg/day to 1 g/kg/day. In other examples, the GABA-receptor agonist is administered in an amount of at least 0.001, 0.005, 0.01, 0.02, 0.05, 0.1, 0.25, 0.5, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 120, 140, 160, 180, 200, 225, 250, 275, 300, 350, 400, 450, 500, 600, 700, 800, 900, 1000, 2000, 5000, 10000, 25000, 50000, or 100000 μg/kg/day. In other examples, the GABA-receptor agonist is administered in an amount of not more than 0.005, 0.01, 0.02, 0.05, 0.1, 0.25, 0.5, 0.75, 1, 2, 3, 4, 5, 6, 7, 8, 9, 10, 15, 20, 25, 30, 35, 40, 45, 50, 55, 60, 65, 70, 75, 80, 85, 90, 95, 100, 120, 140, 160, 180, 200, 225, 250, 275, 300, 350, 400, 450, 500, 600, 700, 800, 900, 1000, 2000, 5000, 10000, 25000, 50000, or 100000 μg/kg/day. In further examples, the GABA-receptor agonist is administered in a dosage range comprising any of the upper and lower limits described herein. In still further examples, the GABA-receptor agonist is administered orally in an amount that is 1-1000 times that of the dosages listed above for intravenous, subcutaneous, intraperitoneal, or intramuscular administration. In particular examples, the GABA-receptor agonist is administered orally in an amount from 1-10,000 mg/kg/day.

In particular embodiments, the GABA-receptor agonist is GABA administered intraperitoneally in an amount of 25-2000 μg/kg/day. In particular examples, the GABA is administered intraperitoneally in amount of 50-1500 μg/kg/day, 100-1000 μg/kg/day, 150-500 μg/kg/day, or 200-400 μg/kg/day. In other embodiments, the GABA-receptor agonist is GABA administered orally in an amount of 100-10,000 mg/kg/day. In particular examples, the GABA is administered orally in amount of 500-5000 mg/kg/day, 1000-4000 mg/kg/day, or 2000-3000 mg/kg/day. In still further embodiments, the GABA-receptor agonist is homotaurine administered orally in an amount of 10-500 mg/kg/day. In particular examples, the homotaurine is administered orally in amount of 12-300 mg/kg/day, 14-200 mg/kg/day, 16-150 mg/kg/day, or 25-100 mg/kg/day.

In certain embodiments, one or both of the immunomodulator compounds and GABA-receptor agonists are administered at a subclinical dose, i.e., a dosage of the immunomodulator or GABA-receptor agonist compound that is less than an effective dosage of the compound when administered as a monotherapy, or at a suboptimal dose, i.e., a dosage of the immunomodulator or GABA-receptor agonist compound that is less than the dosage of that compound that has been found to provide maximum therapeutic benefit when administered as a monotherapy. A suboptimal or subclinical dose of an immunomodulator compound or GABA-receptor agonist is also referred to herein as administration of a “low-dose” of that compound. In some embodiments, the subclinical dose of a compound is about 5%, 10%, 15%, 20%, 25%, 30%, 35%, 40%, 45%, 50%, 55%, 60%, 65%, 70%, or 75% less than the effective dosage of the compound when it is administered as a monotherapy. In other embodiments, the suboptimal dose of a compound is about 5%, 10%, 15%, 20%, 25%, 30%, 35%, 40%, 45%, 50%, 55%, 60%, 65%, 70%, or 75% less than the dosage of that compound that has been found to provide maximum therapeutic benefit when administered as a monotherapy.

In certain embodiments, the GABA-receptor agonist is administered 1, 2, 3, 4, 5, or 6 times per day. In other embodiments, the GABA-receptor agonist is administered every day, every other day, every 3 days, every 4, days, every 5 days, every 6 days, every 7 days, every 8 days, every 9 days, every 10 days, every 11 days, every 12 days, every 13 days, every 14 days, once per week, twice per week, 3 times per week, monthly, twice per month, 3 times per month, or 4 times per month. In further embodiments, the GABA-receptor agonist is administered for 1, 2, 3, 4, 5, 6, 7, 8, 9, 10, 11, 12, 13, or 14 days or for 1, 2, 3, 4, 5, 6, 7, 8, 9, 10, 11, or 12 months or more.

In certain examples, the immunomodulator compound is administered prior to the GABA-receptor agonist. In other examples, the GABA-receptor agonist compound is administered prior to the immunomodulator.

The invention is set forth in more detail in the following illustrative Examples and accompanying Figures, which demonstrate additional attributes and advantages of the invention. The Examples represent select embodiments of the invention and are not limiting.

### EXAMPLES

### Example 1—Combination of Immunomodulator Compound with GABA-Receptor Agonist Enhances Islet β-Cell Content and Hyperglycemia Control in Newly-Diabetic NOD Mice

While immunization with proinsulin/alum and oral GABA restored normoglycemia for a short time, a stronger immunosuppressant was desired to protect residual β-cells and new β-cells (due to β-cell replication, β-cell neogenesis, and/or α-cell transdifferentiation) arising from GABA treatment in NOD mice. The inventors hypothesized that under the protective cover of an immunosuppressant, such as anti-CD3, GABA may be able to promote β-cell replenishment and that these cells could survive, longer-term, in NOD mice.

### Materials and Methods

Newly-diabetic NOD mice (blood glucose 220-300 mg/dL on two consecutive days) were treated with anti-CD3 (non-Fc binding anti-CD3ε F(ab′)₂ from BioExpress, 40 μg/day intravenously for 5 consecutive days) and immediately began one of the following additional treatments:
(1) control PBS IP;
(2) GABA (dissolved in PBS, 250 μg/kg, IP);
(3) control plain drinking water; or
(4a) drinking water containing 6 mg/ml GABA or (4b) drinking water containing 20 mg/ml GABA.
   Mice drink approximately 4 ml/day, so Group 4a consumed approximately 24 mg/day of GABA and Group 4b consumed approximately 80 mg/day of GABA.

Because a relatively high dose of anti-CD3 was administered, almost all of the mice quickly responded to anti-CD3 monotherapy, or combined anti-CD3 plus GABA therapy, and became normoglycemic. This indicates that the treatments quickly preserved residual β-cell mass. However, the residual β-cell mass may have suboptimal function and therefore it was important to assess whether the combination of anti-CD3 plus GABA had a more beneficial effect on islet cells than the immunosuppressant alone.

Three months after beginning treatment, pancreata from the mice were removed and sections (4 μm) were stained with Alexa Fluor 647-anti-glucagon (shown in red in the Figures) and Alexa-488 conjugated with anti-insulin (shown in green in the Figures), followed by staining with 4′,6-diamidino-2-phenylindole (“DAPI”). Images were captured using a fluorescent microscope and at least 30 islets from each group of mice (n=4) were analyzed. Data are representative islet images or expressed as the mean±standard deviation (“SD”) of each group.

### Results

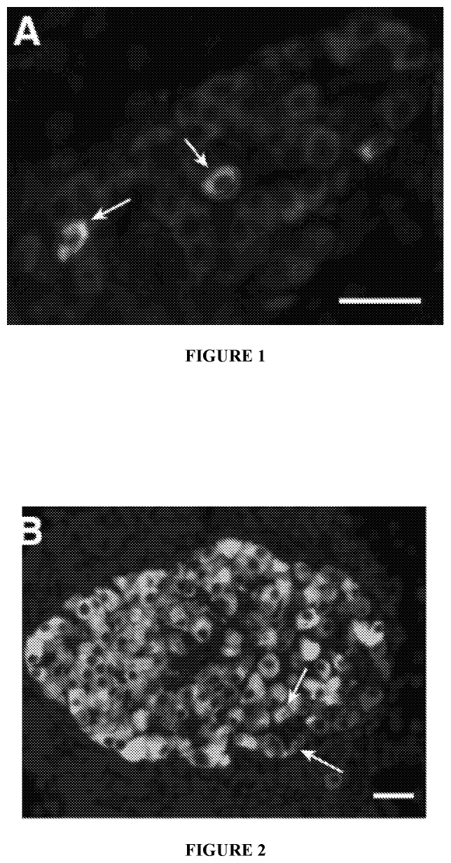

After three months of treatment, it was observed that islets in control diabetic NOD mice, given either anti-CD3 and either plain water or PBS IP, were comprised almost entirely of glucagon-expressing cells with a few bihormonal cells that expressed both insulin and glucagon, and no or very rare β-cells. See FIG. 1. In groups of mice that received anti-CD3 and GABA it was observed that most islets were largely devoid of β-cells, similar to the control groups, but it was also observed that some islets contained many β-cells. The islets that contained many β-cells were always surrounded by a benign insulitis. See FIG. 2.

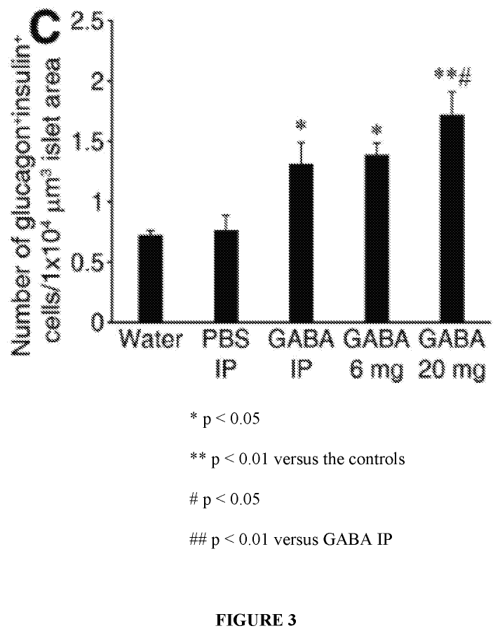

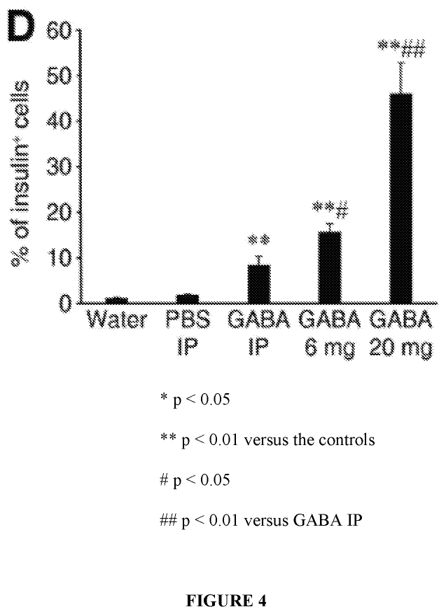

The frequency of bihormonal glucagon⁺ insulin⁺ cells was significantly increased in all GABA-treated mouse groups versus that in control groups. See FIG. 3. The highest GABA dose (20 mg/ml) induced greater numbers of bihormonal cells than that in groups treated with lower dosages of GABA. Id. GABA treatment increased the frequency of insulin⁺ cells per islet, with the highest GABA dose promoting a dramatic 26-fold increase in the frequency of insulin⁺ cells relative to that in the control groups who received anti-CD3 alone. See FIG. 4. Thus, the combination of a GABA receptor agonist and an immunosuppressant had desirable effects on islet cells beyond that provided by treatment with the immunosuppressant alone.

### Discussion

The results indicate that under the cover of immunotherapy, such as by anti-CD3, long-term GABA treatment can help preserve and/or replenish β-cells, leading to increased β-cell content and hyperglycemia control in newly-diabetic NOD mice. Under the cover of anti-CD3 immunotherapy, GABA treatment can also promote a small but significant increase in the number of glucagon+insulin+ islet cells, which may have contributed to the increased β-cell content and hyperglycemia control in newly-diabetic animals. An oral route of GABA administration was much more effective than intraperitoneal administration, which was the route of administration in previous studies with GABA in nonautoimmune mice,² perhaps because of oral GABA's effect on gut immune cells and/or on gut microbiota or the higher GABA dose that was administered orally.

The GABA dose-dependent increase in β-cells/islet may be due to: (1) better protection of residual β-cells, as well as new β-cells or β-like cells, from autoimmune destruction; (2) promoting β-cell replication; (3) promoting α-cell conversion to β-cells; and/or (4) neogenesis of β-cells or α-cells that converted to β-cells. Regardless of the extent to which each of these possible mechanisms may have contributed to the observations, it is clear that in combination with an immunomodulator, treatment with a GABA receptor agonist can lead to increased β-cells in the context of T1D.

### Example 2—Decreased Dose of Immunomodulator with GABA-Receptor Agonist Effectively Ameliorates Hyperglycemia in Newly-Diabetic NOD Mice

Homotaurine is a GABA-Receptor agonist that has >3-fold higher affinity for GABA-R and a longer half-life than GABA in plasma hours vs. 20 minutes for GABA after intravenous or intraperitoneal injection). Based on homotaurine's pharmacokinetic properties and its excellent safety profile, we tested whether homotaurine could be successfully used as the GABA-Receptor agonist in combination with a subclinical dose of immunomodulator.

### Homotaurine Monotherapy Dose-Finding Studies in Newly-Diabetic NOD Mice

Previous studies of GABA monotherapy in newly-diabetic NOD mice showed that this treatment had some ability to temporarily reverse hyperglycemia in newly-diabetic NOD mice. To determine whether homotaurine had therapeutic potential in NOD mice, the inventors performed a dose finding study with homotaurine dissolved in the drinking water at 0, 0.08, 0.25 or 0.75 mg/ml. None of the mouse groups under study differed in their water or food consumption or body weights over the course of the study (data not shown). Newly-diabetic NOD mice that were untreated rapidly progressed to severe hyperglycemia within 1 week. Treatment with homotaurine at 0.08 mg/ml delayed disease progression for a very brief period. Treatment with homotaurine at 0.25 mg/ml restored normoglycemia in all mice. Most of these mice became hyperglycemic again within 6 weeks of treatment, but a few mice displayed extended remission of 14 to 46 weeks (the end of the study). Newly-diabetic mice treated with higher dosage homotaurine (0.75 mg/ml) also displayed some delay in disease progression but this high dosage was on average less effective than the intermediate 0.25 mg/ml dose. Thus, oral homotaurine monotherapy at an appropriate dose can quickly correct hyperglycemia and maintain normoglycemia for a short period, but most of the treated mice become hyperglycemic again.

Combined Low-Dose Homotaurine and Proinsulin/Alum Therapy More Effectively Reverses Hyperglycemia than Either Monotherapy in Newly-Diabetic NOD Mice

Since activation of T-cell GABA-receptors and antigen-based therapy can induce immunoregulatory responses and homotaurine can promote B-cell survival and replication to increase β-cell content, the inventors hypothesized that their combination may have enhanced therapeutic effect for T1D treatment relative to either monotherapy. The inventors had previously determined that proinsulin/alum immunization alone had essentially no therapeutic effect in newly-diabetic NOD mice. Combined treatment with a suboptimal dose of homotaurine (0.08 mg/ml) plus proinsulin/alum lead to an extended average disease reversal time of 24 weeks versus that of 14 weeks for homotaurine monotherapy. Thus, in combination, a low dose of a GABA-R agonist together with an immunomodulator (e.g., proinsulin/alum) had an improved therapeutic effect than optimal dosages of either monotherapy. Future studies will administer proinsulin/alum in combination with GABA.

Combined Treatment with Homotaurine and Low-Dose Anti-CD3 More Effectively Reverses Hyperglycemia in Newly-Diabetic NOD Mice than the Monotherapies

The inventors hypothesized that combining GABA-Receptor activation with an immunosuppressant may allow effective disease reversal using lower dosages of the immunosuppressant and thereby reduce the possibility of side effects. The inventors chose to test anti-CD3 since it is a prototypic immunosuppressant that is in clinical use. In pilot studies, the inventors developed a low-dose anti-CD3 treatment protocol (35 μg anti-CD3 on three consecutive days), which reversed hyperglycemia in about a third of newly-diabetic NOD mice with severe hyperglycemia at entry into the study (blood glucose >350 mg/dL). The inventors then treated newly severely hyperglycemic NOD mice with low-dose anti-CD3 together with homotaurine (0.25 mg/ml), GABA (6 mg/ml), or plain water.

For these studies, the inventors initiated treatment when the NOD mice had blood glucose levels >350 mg/dL. Based on pilot studies, the inventors further reduced the suboptimal anti-CD3 dose described by von Herrath and colleagues,⁷ to three consecutive daily treatments of 35 μg anti-CD3 (hamster anti-CD3ε 2C11 F(ab′)2 fragment, BioXCell, West Lebanon, N.H.) intravenously. At the time of the first anti-CD3 treatment, the animals were randomized to receive plain water, or water containing homotaurine (0.25 mg/ml) or GABA (6 mg/ml), which was continued for the length of the study. Treated mice with two consecutive blood glucose readings below 250 mg/dL were considered to be in remission after which two consecutive blood glucose readings >250 mg/dL was considered to be disease relapse

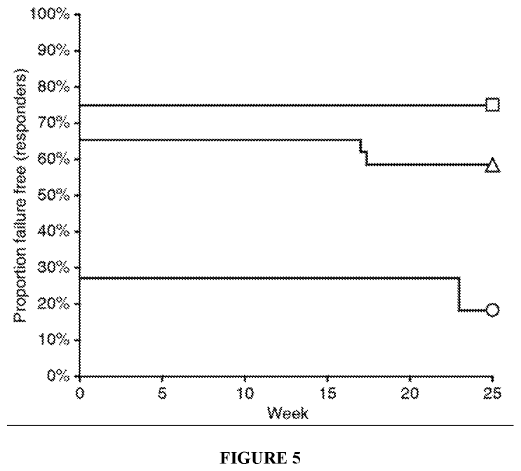

The inventors observed that the addition of either homotaurine or GABA added to the anti-CD3 regimen resulted in significantly greater disease remission than anti-CD3 monotherapy. See Table 1 and FIG. 5. Once the animals went into remission, the vast majority of them stayed normoglycemic for the 25-week post-treatment observation period. See FIG. 5. While a greater percentage of anti-CD3+GABA treated mice responded to therapy compared to those given anti-CD3+homotaurine, there was no statistical difference between these groups. See FIG. 5.

Looking at FIG. 5, data shows percentage of newly-diabetic NOD mice remaining normoglycemic over the ensuing 25-week observation period after receiving low-dose anti-CD3 (FIG. 5, circle symbol), or combined low dose anti-CD3+homoaurine (0.25 mg/ml) (FIG. 5, triangle), or low-dose anti-CD3+GABA (FIG. 5, square). By log rank comparison: anti-CD3 vs. anti-CD3+homotaurine p=0.02; anti-CD3 vs. anti-CD3+GABA p=0.008; and anti-CD3+homotaurine vs. anti-CD3+GABA p=0.35.

Looking at Table 1, newly-diabetic NOD mice received low-dose anti-CD3 or combined low-dose anti-CD3+homotaurine (0.25 mg/ml), or low-dose anti-CD3+GABA. Data shown are the percent of animals in each treatment group that went into remission. Combined therapies were compared to control anti-CD3 monotherapy by Chi-square analysis. There was no statistical difference between anti-CD3+homotaurine vs. anti-CD3+GABA (p=0.55).

TABLE 1
Frequency of disease remission following combined
subclinical-dose anti-CD3 and GABA agonist treatment
vs. low dose anti-CD3 monotherapy

| Group | % responders | p-value |
|---|---|---|
| Anti-CD3 alone | 31% | N/A |
| Anti-CD3 + homotaurine | 64% | 0.05 |
| Anti-CD3 + GABA | 75% | 0.02 |

The inventors observed that while the remission rate was 31% following low-dose anti-CD3 monotherapy, the combination treatment of low-dose anti-CD3+homotaurine had a remission rate of 64%, which was not statistically different from the 75% remission rate following low-dose anti-CD3+GABA. The vast majority of the mice that went into remission following low-dose anti-CD3+homotaurine remained normoglycemic throughout the ensuing months (82% throughout a 25 week observation period). Thus, these combination treatments more than doubled the frequency of disease reversal in mice that had been severely hyperglycemic. It is notable that the increase in remission frequency following combination treatment took place within the first week following treatment. This increased response soon after treatment suggests that homotaurine acted quickly to quell autoimmune responses and/or preserve residual B-cells. Indeed, homotaurine acted quickly to limit B-cell apoptosis immediately following human islet xenografting, and has rapid effects on inflammatory T cell responses in our in vitro assessments. Over the long-term, homotaurine treatment may have also induced α-cell transdifferentiation and neogenesis.

These studies provide evidence for homotaurine's beneficial effects on inflammatory immune responses, β-cell survival and replication, as well as proof-of-principle that homotaurine in combination with immunoregulatory or low-dose immunosuppressive agent can more effectively treat new onset T1D than the respective monotherapies. Thus, the newly-described GABA receptor agonist with immunomodulator combination therapies can be highly effective while using lower doses of the immunomodulator, which should minimize the adverse effects associated with immunomodulatory (in this case, immunosuppressant) use. Withdrawal of GABA or homotaurine can still stabilize the diabetes remission in NOD mice after combination of low dose anti-CD3 and GABA or homotaurine.

### Example 3—Combined Homotaurine with Proinsulin/Alum Therapy More Effectively Reverses Hyperglycemia than Either Monotherapy in Newly-Diabetic NOD Mice

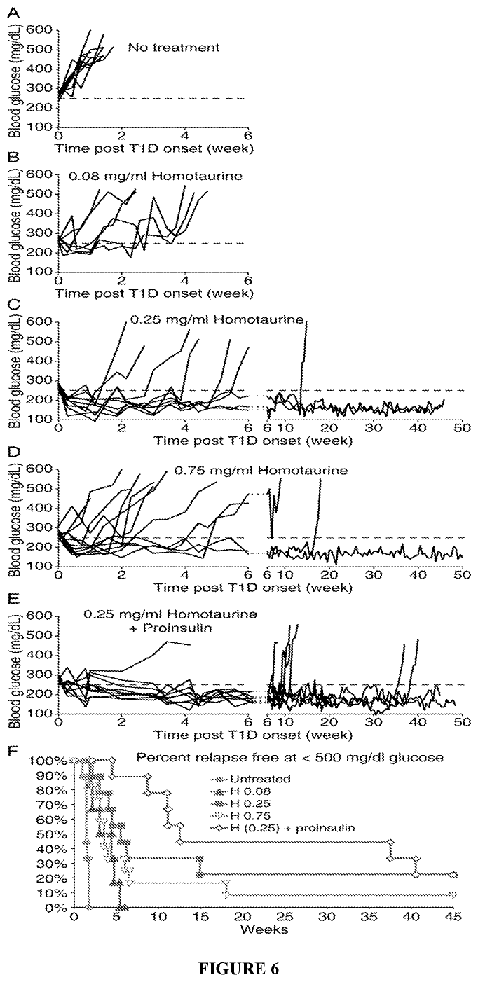

A dose finding study was performed with homotaurine dissolved in the drinking water at 0, 0.08, 0.25 or 0.75 mg/ml to determine the therapeutic potential in NOD mice. None of the mouse groups under study differed in their water or food consumption or body weights over the course of the study (data not shown). Newly-diabetic NOD mice that were untreated rapidly progressed to severe hyperglycemia within 1 week. See FIG. 6A. Treatment with homotaurine at 0.08 mg/ml delayed disease progression for a very brief period (mean of 2.2 weeks). See FIG. 6B. Treatment with homotaurine at 0.25 mg/ml restored normoglycemia in all mice. See FIG. 6C. Most of these mice became hyperglycemic again within 6 weeks of treatment but a few mice displayed extended remission of 14 to 46 weeks (the end of the study), leading to a mean remission period of 14 weeks for all mice. Newly-diabetic mice treated with higher dosage homotaurine (0.75 mg/ml) also displayed some delay in disease progression (see FIG. 6D), but this high dosage was on average less effective that the intermediate 0.25 mg/ml dose. Thus, oral homotaurine monotherapy at an appropriate dose can quickly correct hyperglycemia but most of the treated mice become hyperglycemic again within a short period.

Proinsulin/alum immunization alone was previously shown to have little to no therapeutic effect in newly-diabetic NOD mice. See J. Tian et al., Combined therapy with GABA and proinsulin/alum acts synergistically to restore long-term normoglycemia by modulating T-cell autoimmunity and promoting beta-cell replication in newly-diabetic NOD mice, 63 DIABETES 3128-3134 (2014). We next tested whether the combination of oral homotaurine (0.25 mg/ml) and proinsulin/alum immunization could increase the frequency or length of disease remission in newly diabetic NOD mice. All mice receiving the combination therapy displayed a period of disease remission (see FIG. 6E). The mice receiving the combination therapy had a mean remission period of 24 weeks, which was an increase of 10 weeks over the mean remission period of homotaurine monotherapy, although this difference was not statistically significant. The percentage of relapse-free mice in all groups is shown in FIG. 6F.

### Example 4—Combined Treatment with Homotaurine and Anti-CD3 Reverses Hyperglycemia in Newly-Diabetic NOD Mice

In prior studies, a low-dose anti-CD3 treatment protocol (35 μg anti-CD3 on three consecutive days) was found to reverse hyperglycemia in about a third of newly-diabetic NOD mice with severe hyperglycemia at entry into the study (blood glucose>350 mg/dL).

We tested whether combining homotaurine with anti-CD3 would provide improved results. Newly-severely-hyperglycemic NOD mice were treated with low-dose anti-CD3 and placed on plain water or water containing homotaurine (0.25 mg/ml). A control group received homotaurine alone.

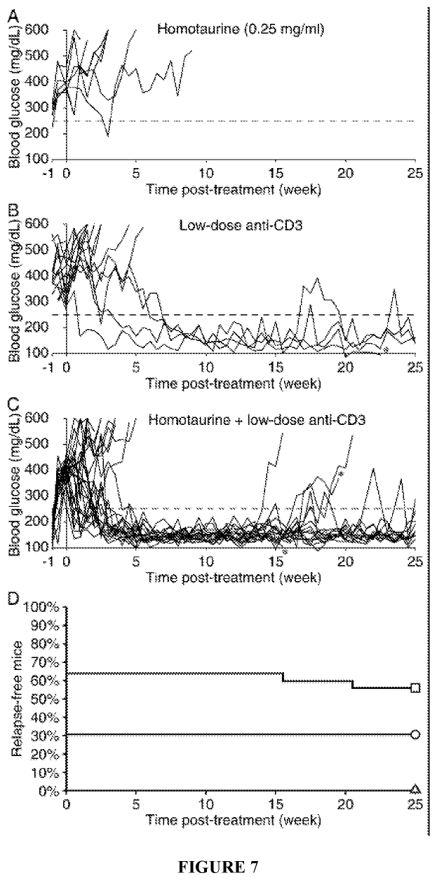

Homotaurine monotherapy was unable to induce remission at this later stage of disease (see FIG. 7A). Low-dose ant-CD3 treatment alone led to disease remission in about 31% of treated animals, although it generally took several weeks for remission to occur (see FIG. 7B, 7D).

The combination of low-dose anti-CD3 and homotaurine doubled the remission rate (to 64%) compared to that of anti-CD3 treatment alone (p=0.05 vs. low-dose anti-CD3 monotherapy) and 82% of these mice remained in remission throughout the 25 week observation period (see FIG. 7C, 7D). Thus, the combination of low-dose anti-CD3 and homotaurine had increased ability to restore and maintain normoglycemia after the development of severe hyperglycemia in NOD mice.

An IPGT test was performed on the mice which remained in remission 25 weeks after initiating treatment. We observed no significant difference between mice that received anti-CD3 alone, and those which received combined therapy, as might be expected since both these groups of mice were normoglycemic. Immunohistological analysis of pancreata from severely diabetic NOD mice that responded to low-dose anti-CD3 monotherapy revealed their islets that had just a few insulin⁺ cells when examined 25 weeks after the initiation of treatment. These nonfunctional islets were essentially insulitis-free. The vast majority of islets in pancreata from mice that had been started on combined therapy 25 weeks earlier were also almost devoid of insulin⁺ cells, except that we observed rare islets that had many B-cells. These functional islets had a surrounding peri-insulitis and islet membrane damage was evident.

### Example 5—Combined Treatment with Homotaurine and Anti-CD3 Increases the Frequency of CD4+ and CD8+ Tregs in the Spleens and Pancreatic Lymph Nodes of Severely Diabetic NOD Mice

Previous studies have shown that treatment with anti-CD3 depletes effector T cells while preserving splenic CD4⁺Foxp3⁺ Tregs in NOD mice (Penaranda, C., Q. Tang, and J. A.

Bluestone. 2011. Anti-CD3 therapy promotes tolerance by selectively depleting pathogenic cells while preserving regulatory T cells. J Immunol 187: 2015-2022). Studies with homotaurine in the EAE mouse model demonstrated that homotaurine monotherapy increased the frequency of splenic CD4⁺Foxp3⁺ and CD8⁺CD122⁺PD-1⁺ Tregs in SJL mice (Tian, J., H. Dang, M. Wallner, R. Olsen, and D. L. Kaufman. 2018. Homotaurine, a safe blood-brain barrier permeable GABAA-R-specific agonist, ameliorates disease in mouse models of multiple sclerosis. Sci Rep 8: 16555). To begin to elucidate the potential mechanisms underlying the therapeutic action of combined homotaurine and anti-CD3 we first characterized the percentages of splenic CD4⁺Foxp3⁺ and CD8⁺CD122⁺PD-1⁺ Tregs in mice 25 weeks after treatment with anti-CD3 alone or combined anti-CD3+homotaurine. We observed that the percentages of splenic CD4⁺Foxp3⁺ and CD8⁺CD122⁺PD-1⁺ Tregs in the mice given combined therapy were significantly higher than that of the mice given anti-CD3 monotherapy (p<0.001 for both). There was no significant difference in the frequency of splenic CD8⁺CD122⁺PD-1⁻ T cells between these two groups of mice. Thus, combined treatment with homotaurine and anti-CD3 significantly increased the frequency of both CD4⁺ and CD8⁺ Tregs in the spleen in comparison to anti-CD3 monotherapy in severely diabetic NOD mice.

Analysis of pancreatic lymph node (PLN) mononuclear cells in additional groups of NOD mice that had been treated with vehicle alone (control), homotaurine alone, low-dose of anti-CD3 alone, or homotaurine+anti-CD3 at 15-18 weeks in age and studied at 3 weeks later showed that treatment with homotaurine or anti-CD3 monotherapies significantly increased the frequencies of CD4⁺CD25⁺Foxp3⁺ and CD8⁺CD122⁺PD-1⁺ cells in the PLN, relative to control groups. Notably, the combination treatment further elevated the average frequencies of CD4⁺CD25⁺Foxp3⁺ cells and CD8⁺CD122⁺PD-1⁺ cells in the PLN above that observed from the monotherapies. This increase was significant for CD4⁺CD25⁺Foxp3⁺ cells in comparison to homotaurine monotherapy. Thus, combined therapy also elevated the average frequencies of CD4⁺ and CD8⁺ Tregs within the target tissue/PLN of NOD mice

### Materials and Methods for Examples 3-5

### T1D Intervention Studies in Newly Diabetic NOD Mice

NOD mice (Taconic Farms, Germantown) were housed in a specific pathogen-free facility. Only female NOD mice were used.

Homotaurine Monotherapy Dose Studies

Blood glucose levels were monitored 2-3 times weekly using a One Touch Ultrasensitive monitor and those with two consecutive daily blood glucose levels between 250-300 mg/dL were entered into the study. The mice were randomly assigned to groups that continually received water containing 0, 0.08, 0.25, or 0.75 mg/ml homotaurine, pH 7.2 through their drinking water. Each mouse consumed on average about 4-5 ml of water per day. Water bottles were changed every five days. Treated mice with two consecutive blood glucose readings below 250 mg/dL were considered to be in remission after which two consecutive blood glucose readings >250 mg/dL was considered disease relapse.

Combined Homotaurine and Proinsulin/Alum Treatment

Newly-diabetic mice (blood glucose 250-300 mg/dL) received 100 μg proinsulin (kindly provided by Eli Lilly, Indianapolis) complexed with alum (Pierce, Rockford, Ill.)) intraperitoneally. The same day, the animals were placed on water containing a low-dose of homotaurine (0.08 mg/ml) which was continued for the length of the study. The mice were immunized once more with proinsulin/alum ten days after the first vaccination. Treated mice were monitored for disease remission and relapse as described above.

Combined Homotaurine and Low-Dose Anti-CD3 Treatment

Treatment was initiated when the NOD mice had blood glucose levels >350 mg/dL. Based on pilot studies, the suboptimal anti-CD3 dose described by von Herrath and colleagues (see D. Bresson et al., Anti-CD3 and nasal proinsulin combination therapy enhances remission from recent-onset autoimmune diabetes by inducing Tregs, 116 J CLIN INVEST 1371-1381 (2006)) was further reduced to three consecutive daily treatments of 35 μg anti-CD3 (hamster anti-CD3ε 2C11 F(ab′)2 fragment, BioXCell, West Lebanon, N.H.) intravenously. At the time of the first anti-CD3 treatment, the animals were randomized to receive plain water, or water containing homotaurine (0.25 mg/ml) or GABA (6 mg/ml) which was continued for the length of the study. Treated mice with two consecutive blood glucose readings below 250 mg/dL were considered to be in remission after which two consecutive blood glucose readings >250 mg/dL was considered to be disease relapse.

### REFERENCES

The invention described herein may be embodied in other specific forms without departing from the spirit or essential characteristics of the invention. The foregoing embodiments are illustrative and not limiting. Additionally, the publications cited herein are incorporated by reference in their entireties for all purposes.
1. J. Tian et al., Combined therapy with GABA and proinsulin/alum acts synergistically to restore long-term normoglycemia by modulating T-cell autoimmunity and promoting beta-cell replication in newly diabetic NOD mice, 63 DIABETES 3128-3134 (2014).
2. N. Ben-Othman et al., Long-Term GABA Administration Induces Alpha Cell-Mediated Beta-like Cell Neogenesis, 168 Cell 73-85 (2017).
3. K. C. Herold et al., Anti-CD3 monoclonal antibody in new-onset type 1 diabetes mellitus, 346 NEW ENGLAND JOURNAL OF MEDICINE 1692-1698 (2002).
4. B. Keymeulen et al., Insulin needs after CD3-antibody therapy in new-onset type 1 diabetes, 352 NEW ENGLAND JOURNAL OF MEDICINE 2598-2608 (2005).
5. T. P. Staeva et al., Recent lessons learned from prevention and recent-onset type 1 diabetes immunotherapy trials, 62 DIABETES 9-17 (2012).
6. K. C. Herold et al., Ab ATEST: Teplizumab (anti-CD3 mAb) treatment preserves C-peptide responses in patients with new-onset type 1 diabetes in a randomized controlled trial: metabolic and immunologic features at baseline identify a subgroup of responders, 62 DIABETES 3766-3774 (2013).
7. D. Bresson et al., Anti-CD3 and nasal proinsulin combination therapy enhances remission from recent-onset autoimmune diabetes by inducing Tregs., 116 J CLIN INVEST 1371-1381 (2006).

1. A method of treating inflammatory disease in a human or animal subject in need thereof, the method comprising:
administering to the human or animal subject one or more immunomodulator compounds and one or more GABA-receptor agonists in an amount effective ameliorate said inflammatory disease.

2. The method of claim 1, wherein the one or more immunomodulator compounds and one or more GABA-receptor agonists are administered in an amount effective to reduce inflammation.

3. The method of claim 1, wherein one or both of the immunomodulator compounds and GABA-receptor agonists are administered at a dosage that is less than an effective dosage of the compound when administered as a monotherapy.

4. The method of claim 1, wherein the inflammatory disease is selected from the group consisting of type-1 diabetes, rheumatoid arthritis, and multiple sclerosis.

5. The method of claim 4, wherein the inflammatory disease is type-1 diabetes.

6. The method of claim 1, wherein the one or more immunomodulator compounds and one or more GABA-receptor agonists are administered in an amount effective to prevent, reduce, and/or treat hyperglycemia in the human or animal subject.

7. The method of claim 1, wherein the one or more immunomodulator compounds comprise one or more immunostimulants or immunosuppressants.

8. The method of claim 1, wherein the one or more immunomodulator compounds comprise one or more immunosuppressants.

9. The method of claim 1, wherein the one or more immunomodulator compounds are selected from the group consisting of an anti-CD3 immunotherapy compound, corticosteroids, prednisone, budesonide, prednisolone, methylprednisolone, calcineurin inhibitors, cyclosporine, tacrolimus, mTOR inhibitors, sirolimus, everolimus, IMDH inhibitors, azathioprine, leflunomide, mycophenolate, biologics, abatacept, adalimumab, anakinra, certolizumab, etanercept, golimumab, infliximab, ixekizumab, natalizumab, rituximab, secukinumab, tocilizumab, ustekinumab, vedolizumab, monoclonal antibodies, basiliximab, daclizumab, muromonab, anti-lymphocyte globin, anti-thymocyte globin, lymphocyte immune globulin, thymoglobulin, mycophenolate mofetil, mycophenolate sodium, glucocorticoids, aspirin, celecoxib, diclofenac, diflunisal, etodolac, ibuprofen, indomethacin, ketoprofen, ketorolac, nabumetone, naproxen, oxaprozin, piroxicam, salsalate, sulindac, and tolmetin, methotrexate, hydroxychloroquine, sulfasalazine, copaxone, and interferon β.

10. The method of claim 1, wherein the one or more immunomodulator compounds comprise an anti-CD3 immunotherapy compound.

11. The method of claim 10, wherein the anti-CD3 immunotherapy compound comprises non-Fc binding anti-CD3ε Fab.

12. The method of claim 1, wherein the GABA-receptor agonist is selected from the group consisting of thiopental, thiamylal, pentobarbital, secobarbital, hexobarbital, butobarbital, amobarbital, barbital, mephobarbital, phenobarbital, primidone, midazolam, triazolam, lometazepam, flutazolam, nitrazepam, fluritrazepam, nimetazepam, diazepam, medazepam, oxazolam, prazeam, tofisopam, rilmazafonoe, lorazepam, temazepam, oxazepam, fluidazepam, chlordizaepoxide, cloxazolam, flutoprazepam, alprazolam, estazolam, bromazepam, flurazepam, clorazepate potassium, haloxazolam, ethyl loflazepate, qazepam, clonazepam, mexazolam, etizolam, brotizolam, clotizaepam, propofol, fospropofol, zolpidem, zopiclone, exzopiclone, muscimol, THIP/gaboxadol, Isoguvacine, Kojic amine, GABA, Homotaurine, Homohypotaurine, Trans-aminocyclopentane-3-carboxylic acid, Trans-amino-4-crotonic acid, β-guanidinopropionic acid, homo-β-proline, Isonipecotic acid, 3-((aminoiminomethyl)thio)-2-propenoic acid (ZAPA), Imidazoleacetic acid, and piperidine-4-sulfonic acid (P4S).

13. The method of claim 12, wherein the GABA-receptor agonist is GABA.

14. The method of claim 1, wherein said immunomodulator compound is administered prior to said GABA-receptor agonist.

15. The method of claim 1, wherein said GABA-receptor agonist compound is administered prior to said immunomodulator.

16. The method of claim 1, wherein said GABA-receptor agonist and said immunomodulator compound are each individually administered either orally, subcutaneously, intramuscularly, or intraperitoneally.

17. The method of claim 1, wherein said GABA-receptor agonist and said immunomodulator compound are administered orally.

18. The method of claim 1, wherein said GABA-receptor agonist and said immunomodulator compound are administered subcutaneously, intramuscularly, or intraperitoneally.
